(19) United States
(12) Patent Application Publication
(10) Pub. No.: US 2003/0003223 A1
(43) Pub. Date: Jan. 2, 2003

(54) Methods and compositions for binding histidine-containing proteins to substrates

(75) Inventors: Daniel E. Morse, Santa Barbara, CA (US); Galen D. Stucky, Goleta, CA (US); John H. Gaul, Los Altos, CA (US)

Correspondence Address: David Maher, McCutchen, Doyle, Brown & Enersen, LLP, 18th Floor, Three Embarcadero Center, San Francisco, CA, 94111, US

(73) Assignee: The Regents of the University of California

(21) Appl. No.: 10/118,865

(22) Filed: Apr. 8, 2002

Related U.S. Application Data
(60) Provisional application No. 60/282,433, filed on Apr. 7, 2001.

(51) Int. Cl. C07F 7/02; G01N 33/53; G01N 33/542; C07F 7/04; B05D 3/00

(52) U.S. Cl. 427/2.11; 556/400; 556/9; 556/413; 435/7.9

(57) ABSTRACT

Methods, compositions and articles of manufacture for binding histidine containing proteins to substrates are provided. A substrate having reactive groups is contacted with a substrate modifier comprising a silane, a linker, and an active site to form an activated substrate. The activated substrate is then reacted with a reagent that binds to the active site and comprises a ligand that can bind to a metal ion to form a chelator which is then chelated to a metal ion to form a metal-chelated substrate. A histidine-containing protein having an arrangement of histidine residues that can bind to two available cis valencies on the chelated metal ion is then incubated with the metal-chelated substrate to form a protein-substrate complex. The protein can be deposited in a pattern through any suitable technique. The protein is bound in an active form allowing it to perform native functions, including enzymatic functions. In one aspect, the protein is a silicatein that can incorporate optionally derivatized silicas and/or silicones onto the substrate. The methods can be used in multiplex form to deposit pluralities of different proteins on a substrate. Sensors, biocatalysts and microfluidic devices incorporating such protein-substrate complexes are also provided. Kits comprising reagents for performing such methods are also provided.

### RELATED APPLICATION DATA

This application claims the benefit of provisional application Serial No. 60/282,433 filed Apr. 7, 2001, the entire disclosure of which is herein incorporated by reference.

### TECHNICAL FIELD

This invention relates to methods, articles and compositions for derivatizing substrates and for binding proteins thereto.

### BACKGROUND OF THE INVENTION

Nature has devised many ways in which to place proteins at precise locations. The ultrastructure of organisms, cell migration, development, formation of cytological structure and intracellular trafficking all rely on the controlled localization of proteins for their proper occurrence. In biological formation of structures, controlled localization of proteins can lead to elegant designs. The huge variety of diatom structures created under ambient conditions is an example of the exquisite results that can be achieved through control over protein localization.

Efforts by man to imitate nature in localizing proteins have been relatively crude and limited. However, advances in cellular and molecular biology have revealed a number of ways in which proteins are targeted to specific intracellular and extracellular locations. Scientists have been able to utilize the targeting signals identified in such experiments to target proteins to new locations inside the cell and within the organism.

Proteins have previously been attached in bulk to a number of matrices such as agarose. But many known techniques for attaching proteins to substrates result in the inactivation of much of the protein used. This is disadvantageous where the function of the protein and its normal interactions with other molecules is to be studied or utilized, particularly where the amount of protein available is costly or limited. Furthermore, known methods can be limited in the degree of surface derivatization achieved. Additionally, in many instances it is desirable to be able to place proteins in particular locations rather than randomly, for example in forming protein arrays.

There is a need in the art for methods of localizing proteins onto substrates, and for devices, compositions and articles of manufacture useful in such methods.

### BRIEF DESCRIPTION OF THE DRAWINGS

FIG. 1 shows a fluorescent image of silica deposited on a substrate through histidine-tagged silicatein alpha protein bound to the substrate via the bis-carboxymethyl-L-lysine (BCML) route described in Example 1. Cover slips were reacted to produce nickel-chelates on their surface. Histidine-tagged silicatein alpha was incubated with the coverslips in a Tris buffer (25 mM Tris pH 7.0, 50 mM NaCl, 0.1 wt. % CHAPS). The cover slips were then rinsed with buffer and placed in a tube containing 0.75 mls tetraethoxysilane (TEOS) with 0.45 mls of buffer (containing 2.5 uM PDMPO, a fluorescent substrate that can be incorporated into silica by silicateins). Long exposures of the activated substrates to BCML were found to be disadvantageous.

FIG. 2 shows a nonfluorescent image of the substrate in FIG. 1, demonstrating silica growth.

FIGS. 3 and 4 show nonfluorescent images of glass slide substrates treated similarly to the cover slips in FIGS. 1 and 2, using a histidine tagged silicatein beta rather than silicatein alpha. FIG. 4 differs in that the silicatein beta was heat treated prior to incubation with the substrate, possibly accounting for the coarser distribution seen in FIG. 4. FIG. 5 is a negative control which was not incubated with silicatein beta.

FIGS. 6-9 show fluorescent images of red fluorescent protein (RFP) bound to silica glass substrates by similar techniques. Histidine-tagged RFP (a mutant version of green fluorescent protein) was bound to glass slides prepared by the general method described in Example 1. The RFP was found to have coagulated into fibers after rinsing the slides with methanol, in vacuo drying, and cold storage.

FIGS. 10-12 show optical images of silica growth produced on a patterned surface by silicatein alpha linked to the surface by the BCML route using a nickel chelate. The patterns were formed using a rectangular electron beam patterned mold on a silicon chip.

FIGS. 13-17 show patterned attachment of RFP to a silicon substrate via a nickel chelate formed using the BCML linker method. TEM grid molds were used to prepare patterned silane, which was then stamped onto a silicon chip, which was then heated for 10 minutes at 60° C. In some cases two different silanes were used in two stamping steps. A 40 mmol solution of Z6040 in ethanol was found to be best for producing a pattern in this experiment.

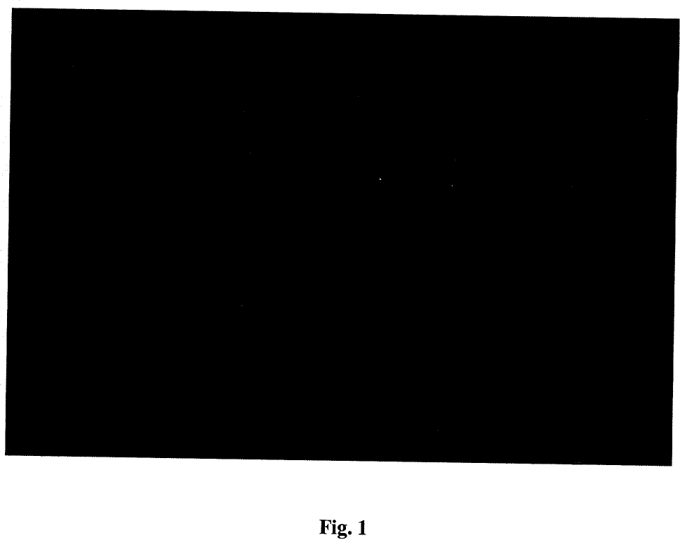

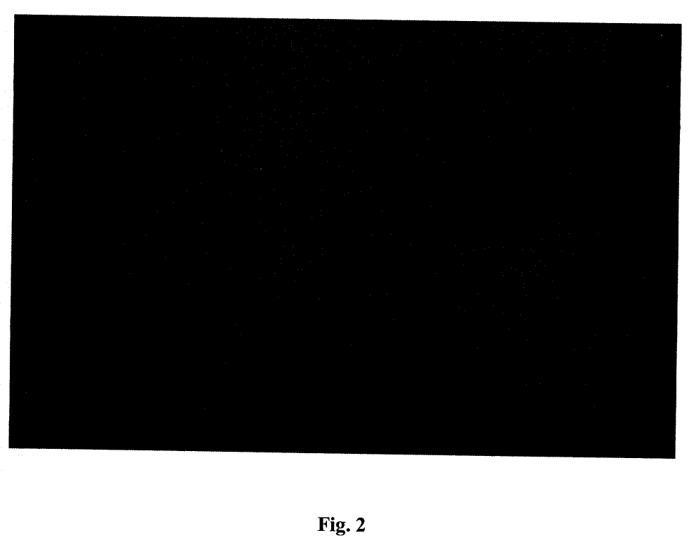

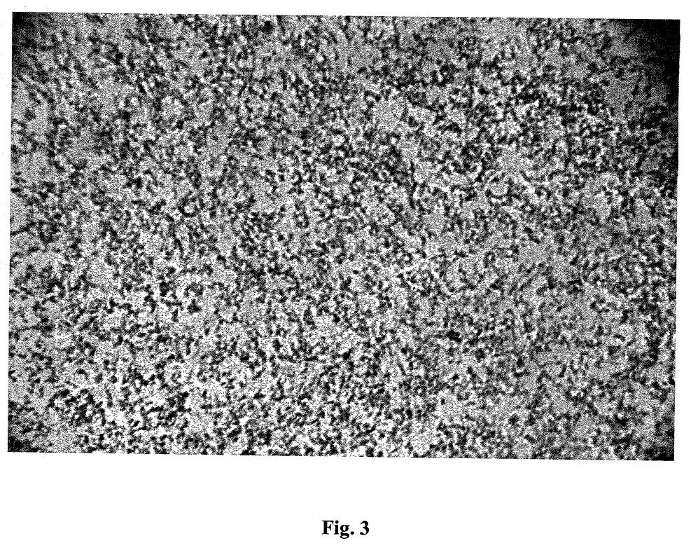

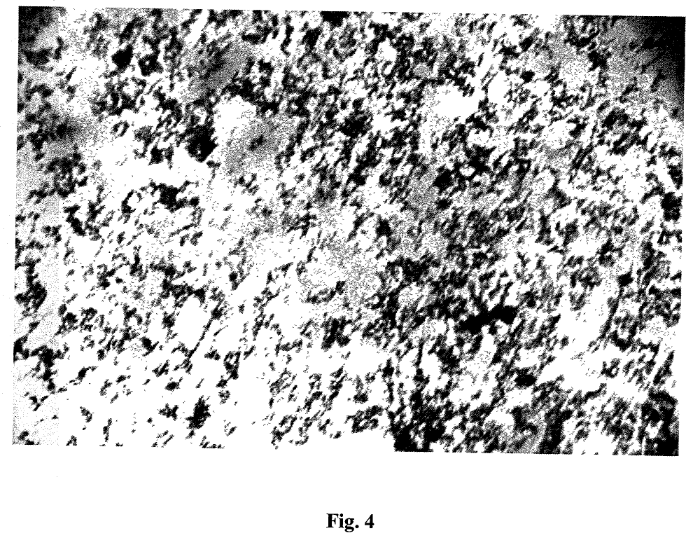

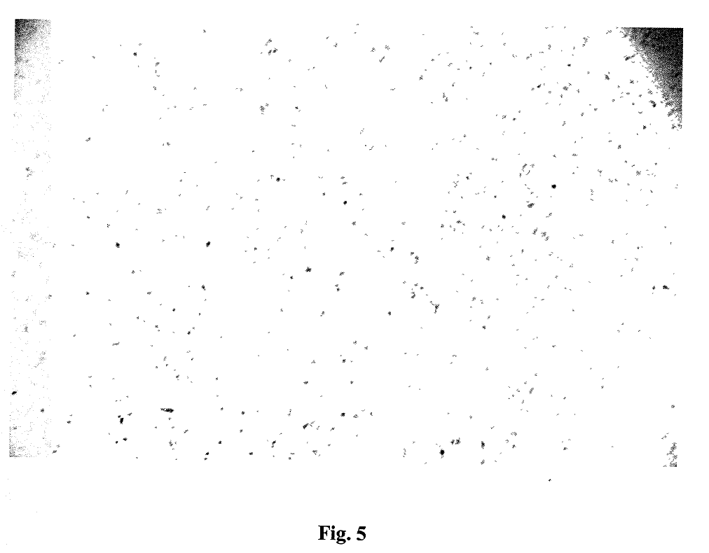

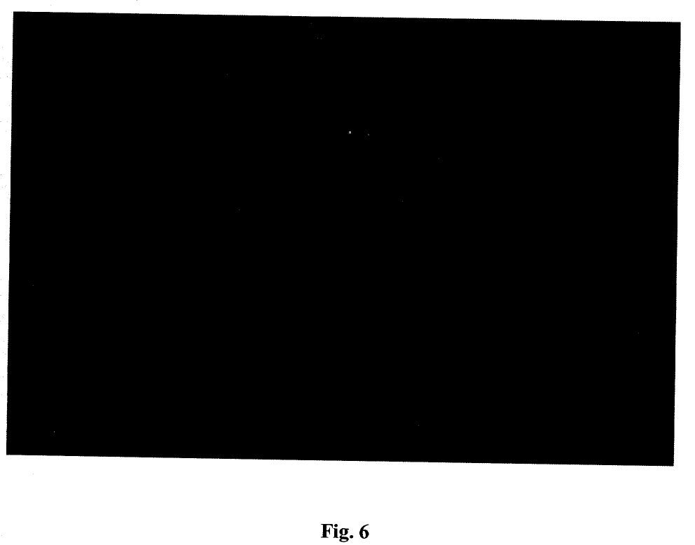

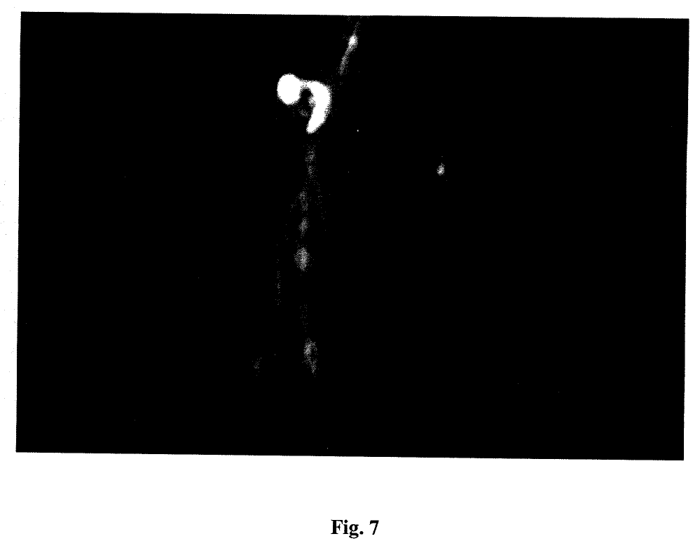

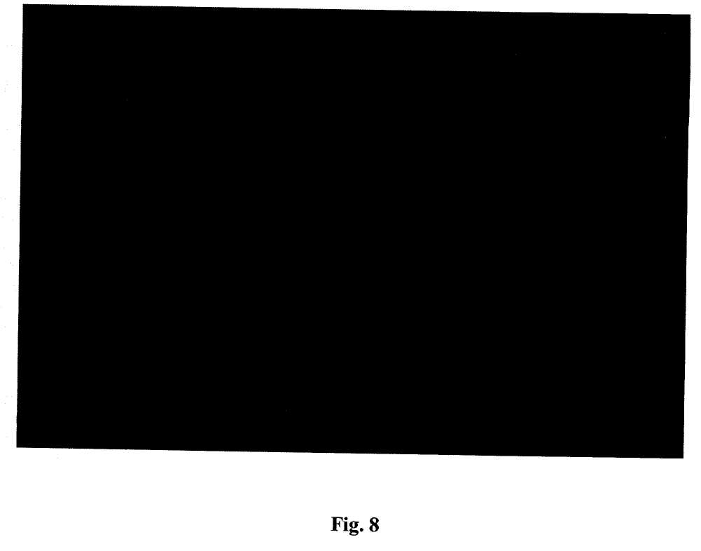

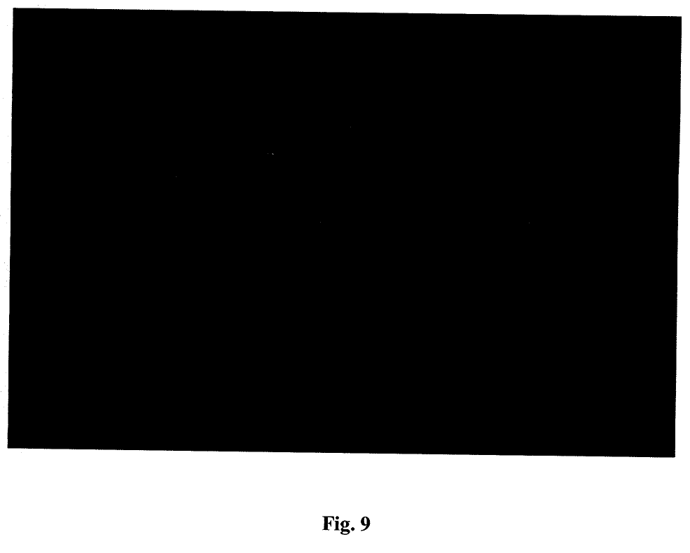

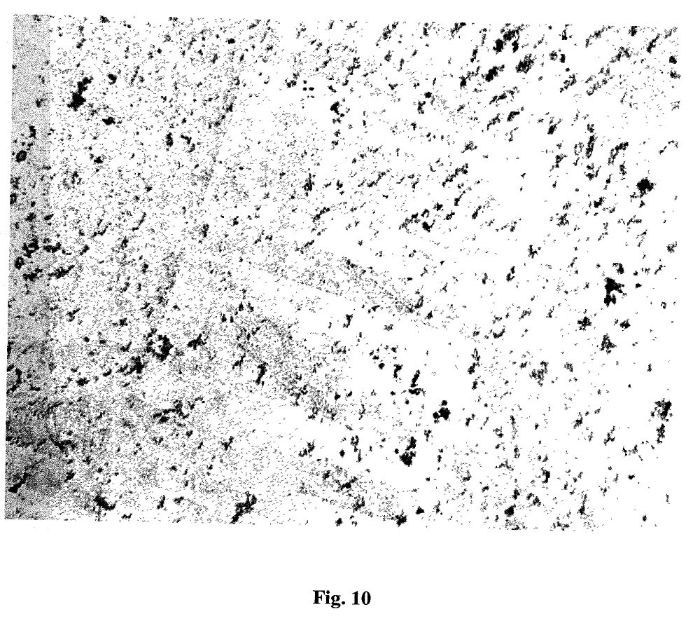

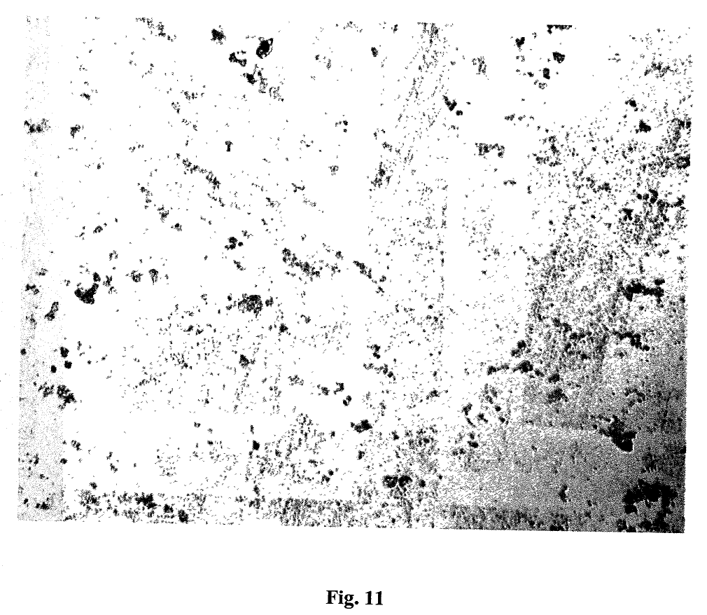

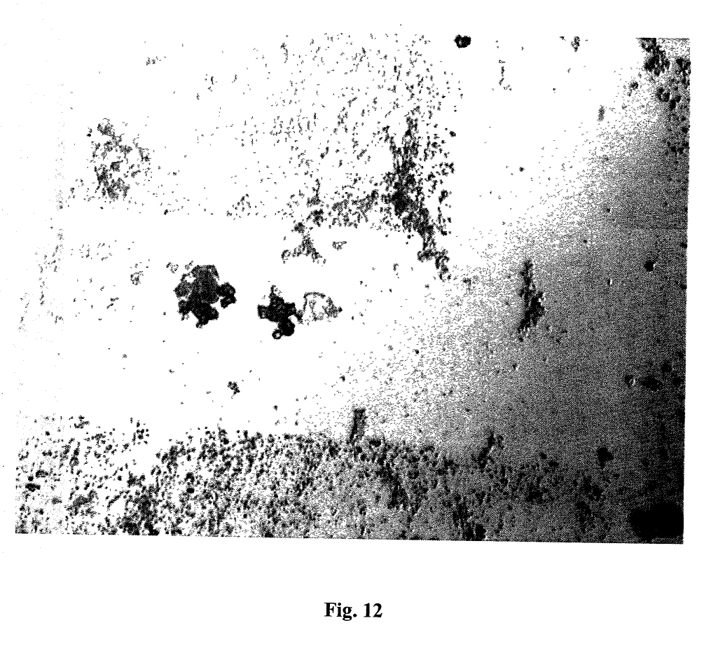

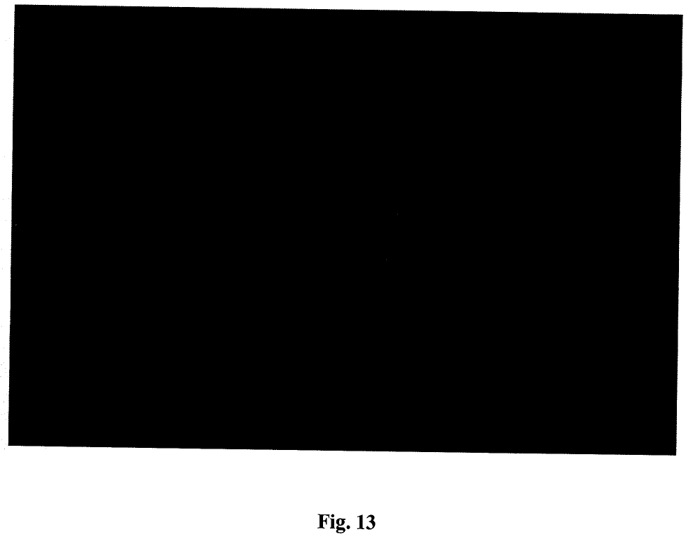

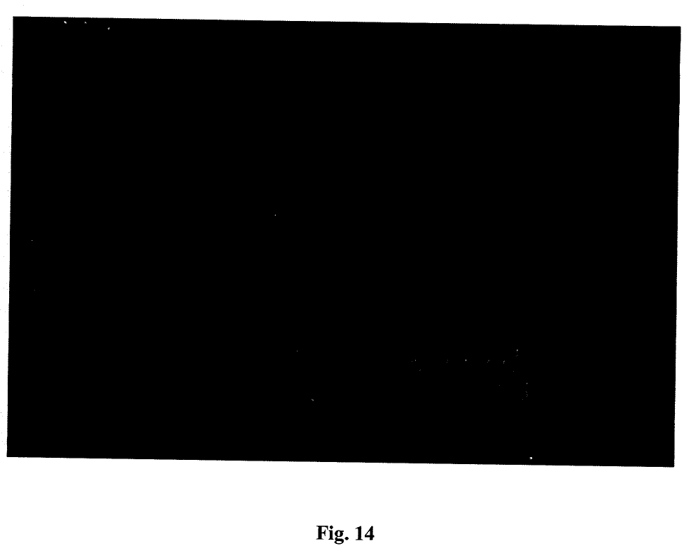

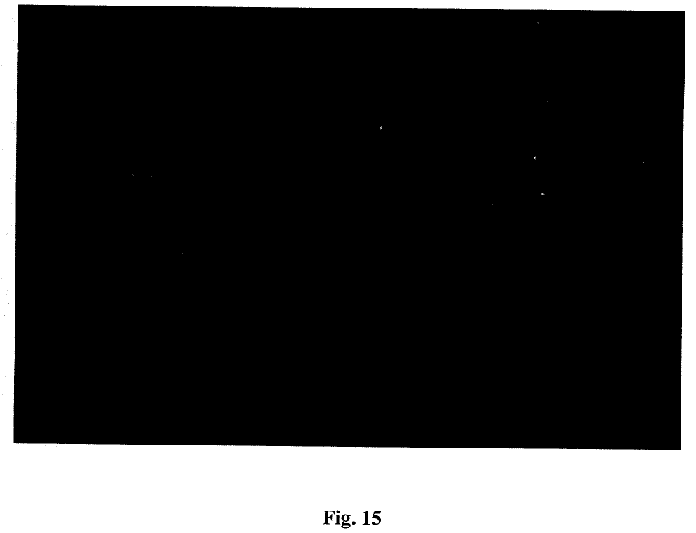

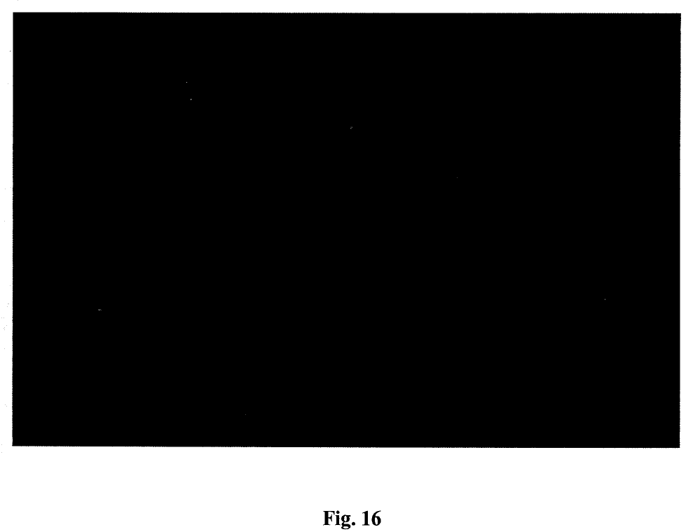

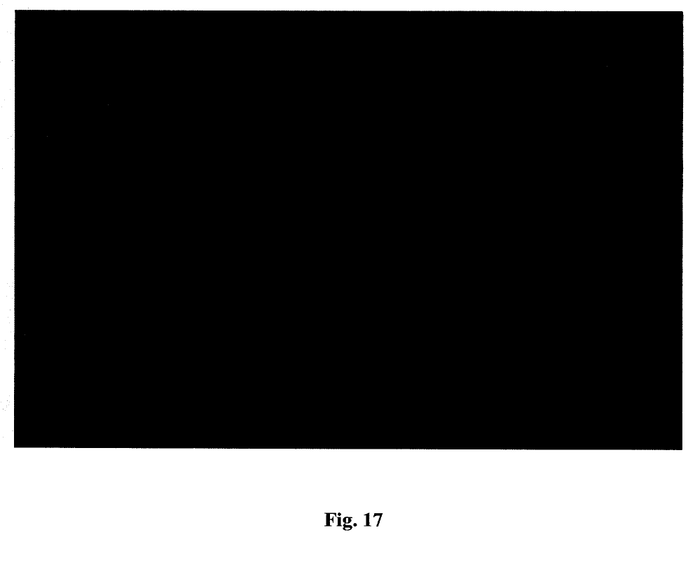

### DETAILED DESCRIPTION OF THE INVENTION

The attachments of proteins to a variety of substrates has great potential in applications such as sensors, biocatalysts, or as “instruments on a chip.” This is especially true in the situation where arrays of proteins can be created or when the protein can be attached to mesoporous substrates such as mesoporous silica. Nickel, cobalt and copper NTA complexes are known to bind 6×-His tagged proteins and are used commercially on agarose and other substrates for protein purification.

The invention provides multistep methods in which chelating groups can be added to substrates in order to bind histidine-containing proteins to the substrate. These multistep methods allow for a high degree of control during synthesis, and permit production of substrates having a very high coverage of their surface area with chelating groups. Such high degrees of control and of surface derivatization have not previously been available. The methods advantageously employ reagents which form a final linker between the chelating group and the substrate that is resistant to reducing conditions, and can be used in the presence of mercaptans and other reducing agents without destroying the linker and releasing the histidine-containing protein.

The methods have application in any technique where catalysts or supports for enzymes are used, for example in the synthesis of pharmaceuticals, nutrients, nutraceuticals, fine chemicals, hydrocarbon cracking, and in energy production and transduction. The methods can also be used to create nanoporous or mesoporous silicas which can be surfactant or copolymer templated and of hexagonal or cubic geometry. The methods can be used to create templates for controlling polymerization of materials, and to form waveguides for photonic applications. The methods can be used for controlling structure and placement at different length scales, in electronic and photonic applications, in the separation of materials, and in microfluidics.

The methods can be used in the formation of sensors, biocatalysts, instruments on a chip, protein arrays, protein chips, biochips, biosensors, and catalysts. Arrays of catalysts can be anchored on a substrate via the methods of the invention, and can be used for assembly line catalysis when deposited in a specific pattern or for bulk catalysis when deposited on a high surface area material like silica or other metallooxides.

Biosensors can be prepared in which proteins such as receptor proteins, antibodies, enzymes, ion channel proteins, and signal transducing proteins are attached to a sensing device via the methods of the invention and used to detect the binding of a specific analyte whose presence is to be detected and quantified. Proteins can be attached to the surface of a CCD chip, for example, via the methods of the invention where the proteins directly or indirectly emit, can be made to emit, or are linked to a molecule that emits, light.

Multiplex methods are provided employing 2, 3, 4, 5, 10 15, 20, 25, 50, 100, 200, 500, 1000 or more different proteins on a substrate. Also provided is an article formed by the reaction of a substrate having active oxygen atoms, active hydroxyl groups, or a combination thereof, and a substrate modifier of the general formula N,N′-bis-(trialkoxysilylalkyl)ethylene diamine. Also provided are the substrate modifier itself, as well as the N,N′-biscarboxymethylated form. Kits comprising components useful for such methods are also provided.

Before the present invention is described in detail, it is to be understood that this invention is not limited to the particular methodology, devices, solutions or apparatuses described, as such methods, devices, solutions or apparatuses can, of course, vary. It is also to be understood that the terminology used herein is for the purpose of describing particular embodiments only, and is not intended to limit the scope of the present invention.

Use of the singular forms “a,” “an,” and “the” include plural references unless the context clearly dictates otherwise. Thus, for example, reference to “a protein” includes a plurality of proteins, reference to “a substrate” includes a plurality of such substrates, reference to “a chelator” includes a plurality of chelators, and the like.

Terms such as “connected,” “attached,” “linked,” and “conjugated” are used interchangeably herein and encompass direct as well as indirect connection, attachment, linkage or conjugation unless the context clearly dictates otherwise. Where a range of values is recited, it is to be understood that each intervening integer value, and each fraction thereof, between the recited upper and lower limits of that range is also specifically disclosed. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. For any element of an invention for which a plurality of options are disclosed, examples of that invention in which each of those options is individually excluded along with all possible combinations of excluded options are hereby disclosed.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

Definitions

In describing the present invention, the following terms will be employed, and are intended to be defined as indicated below.

“Alkyl” refers to a branched, unbranched or cyclic saturated hydrocarbon group of 1 to 24 carbon atoms optionally substituted at one or more positions, and includes polycyclic compounds. Examples of alkyl groups include optionally substituted methyl, ethyl, n-propyl, isopropyl, n-butyl, s-butyl, t-butyl, n-amyl, isoamyl, n-hexyl, n-heptyl, n-octyl, n-decyl, hexyloctyl, tetradecyl, hexadecyl, eicosyl, tetracosyl and the like, as well as cycloalkyl groups such as cyclopropyl, cyclobutyl, cyclopentyl, cyclohexyl, cycloheptyl, cyclooctyl, adamantyl, and norbornyl. The term “lower alkyl” refers to an alkyl group of 1 to 6 carbon atoms, preferably 1 to 4 carbon atoms. Exemplary substituents on substituted alkyl groups include hydroxyl, cyano, alkoxy, ═O, ═S, —NO₂, halogen, haloalkyl, heteroalkyl, amine, thioether and —SH.

“Alkoxy” refers to an “—Oalkyl” group, where alkyl is as defined above. A “lower alkoxy” group intends an alkoxy group containing one to six, more preferably one to four, carbon atoms.

“Alkenyl” refers to a branched, unbranched or cyclic hydrocarbon group of 2 to 24 carbon atoms containing at least one carbon-carbon double bond optionally substituted at one or more positions. Examples of alkenyl groups include ethenyl, 1-propenyl, 2-propenyl (allyl), 1-methylvinyl, cyclopropenyl, 1-butenyl, 2-butenyl, isobutenyl, 1,4-butadienyl, cyclobutenyl, 1-methylbut-2-enyl, 2-methylbut-2-en-4-yl, prenyl, pent-1-enyl, pent-3-enyl, 1,1-dimethylallyl, cyclopentenyl, hex-2-enyl, 1-methyl-1-ethylallyl, cyclohexenyl, heptenyl, cycloheptenyl, octenyl, cyclooctenyl, decenyl, tetradecenyl, hexadecenyl, eicosenyl, tetracosenyl and the like.

Preferred alkenyl groups herein contain 2 to 12 carbon atoms. The term “lower alkenyl” intends an alkenyl group of 2 to 6 carbon atoms, preferably 2 to 4 carbon atoms. The term “cycloalkenyl” intends a cyclic alkenyl group of 3 to 8, preferably 5 or 6, carbon atoms. Exemplary substituents on substituted alkenyl groups include hydroxyl, cyano, alkoxy, ═O, ═S, —NO₂, halogen, haloalkyl, heteroalkyl, amine, thioether and —SH.

“Alkenyloxy” refers to an “—Oalkenyl” group, wherein alkenyl is as defined above.

“Alkylaryl” refers to an alkyl group that is covalently joined to an aryl group. Preferably, the alkyl is a lower alkyl. Exemplary alkylaryl groups include benzyl, phenethyl, phenopropyl, 1-benzylethyl, phenobutyl, 2-benzylpropyl and the like.

“Alkylaryloxy” refers to an “—Oalkylaryl” group, where alkylaryl is as defined above.

“Alkynyl” refers to a branched or unbranched hydrocarbon group of 2 to 24 carbon atoms containing at least one —C≡C— bond, optionally substituted at one or more positions. Examples of alkynyl groups include ethynyl, n-propynyl, isopropynyl, propargyl, but-2-ynyl, 3-methylbut-1-ynyl, octynyl, decynyl and the like. Preferred alkynyl groups herein contain 2 to 12 carbon atoms. The term “lower alkynyl” intends an alkynyl group of 2 to 6, preferably 2 to 4, carbon atoms, and one —C≡C— bond. Exemplary substituents on substituted alkynyl groups include hydroxyl, cyano, alkoxy, ═O, ═S, —NO₂, halogen, haloalkyl, heteroalkyl, amine, thioether and —SH.

“Amide” refers to —C(O)NHR, where R is alkyl, aryl, alkylaryl or hydrogen.

“Amine” refers to an —N(R′)R″ group, where R′ and R″ are independently selected from hydrogen, alkyl, aryl, and alkylaryl.

“Aryl” refers to an aromatic group which has at least one ring having a conjugated pi electron system and includes carbocyclic, heterocyclic and polycyclic aryl groups, and can be optionally substituted at one or more positions. Typical aryl groups contain 1 to 5 aromatic rings, which may be fused and/or linked. Exemplary aryl groups include phenyl, furanyl, azolyl, thiofuranyl, pyridyl, pyrimidyl, pyrazinyl, triazinyl, indenyl, benzofuranyl, indolyl, naphthyl, quinolinyl, isoquinolinyl, quinazolinyl, pyridopyridinyl, pyrrolopyridinyl, purinyl, tetralinyl and the like. Exemplary substituents on optionally substituted aryl groups include alkyl, alkoxy, alkylcarboxy, alkenyl, alkenyloxy, alkenylcarboxy, aryl, aryloxy, alkylaryl, alkylaryloxy, fused saturated or unsaturated optionally substituted rings, halogen, haloalkyl, heteroalkyl, —S(O)R, sulfonyl, —SO₃R, —SR, —NO₂, —NRR′, —OH, —CN, —C(O)R, —OC(O)R, —NHC(O)R, —(CH2)ₙCO₂R or —(CH2)ₙCONRR′ where n is 0-4, and wherein R and R′ are independently H, alkyl, aryl or alkylaryl.

“Aryloxy” refers to an “—Oaryl” group, where aryl is as defined above.

“Carbocyclic” refers to an optionally substituted compound containing at least one ring and wherein all ring atoms are carbon, and can be saturated or unsaturated.

“Carbocyclic aryl” refers to an optionally substituted aryl group wherein the ring atoms are carbon.

“Halo” or “halogen” refers to fluoro, chloro, bromo or iodo. Of the halogens, chloro and fluoro are generally preferred.

“Haloalkyl” refers to an alkyl group substituted at one or more positions with a halogen, and includes alkyl groups substituted with only one type of halogen atom as well as alkyl groups substituted with a mixture of different types of halogen atoms. Exemplary haloalkyl groups include trihalomethyl groups, for example trifluoromethyl.

“Heteroalkyl” refers to an alkyl group wherein one or more carbon atoms and associated hydrogen atom(s) are replaced by an optionally substituted heteroatom, and includes alkyl groups substituted with only one type of heteroatom as well as alkyl groups substituted with a mixture of different types of heteroatoms. Heteroatoms include oxygen, sulfur, and nitrogen. As used herein, nitrogen heteroatoms and sulfur heteroatoms include any oxidized form of nitrogen and sulfur, and any form of nitrogen having four covalent bonds including protonated forms. An optionally substituted heteroatom refers to replacement of one or more hydrogens attached to a nitrogen atom with alkyl, aryl, alkylaryl or hydroxyl.

“Heterocyclic” refers to a compound containing at least one saturated or unsaturated ring having at least one heteroatom and optionally substituted at one or more positions. Typical heterocyclic groups contain 1 to 5 rings, which may be fused and/or linked, where the rings each contain five or six atoms. Examples of heterocyclic groups include piperidinyl, morpholinyl and pyrrolidinyl. Exemplary substituents for optionally substituted heterocyclic groups are as for alkyl and aryl at ring carbons and as for heteroalkyl at heteroatoms.

“Heterocyclic aryl” refers to an aryl group having at least 1 heteroatom in at least one aromatic rings. Exemplary heterocyclic aryl groups include furanyl, thienyl, pyridyl, pyridazinyl, pyrrolyl, N-lower alkyl-pyrrolo, pyrimidyl, pyrazinyl, triazinyl, tetrazinyl, triazolyl, tetrazolyl, imidazolyl, bipyridyl, tripyridyl, tetrapyridyl, phenazinyl, phenanthrolinyl, purinyl and the like.

“Hydrocarbyl” refers to hydrocarbyl substituents containing 1 to about 20 carbon atoms, including branched, unbranched and cyclic species as well as saturated and unsaturated species, for example alkyl groups, alkylidenyl groups, alkenyl groups, alkylaryl groups, aryl groups, and the like. The term “lower hydrocarbyl” intends a hydrocarbyl group of one to six carbon atoms, preferably one to four carbon atoms.

“Optional” or “optionally” means that the subsequently described event or circumstance may or may not occur, and that the description includes instances where said event or circumstance occurs singly or multiply and instances where it does not occur at all. For example, the phrase “optionally substituted alkylene” means an alkylene moiety that may or may not be substituted and the description includes both unsubstituted, monosubstituted, and polysubstituted alkylenes.

A “substituent” refers to a group that replaces one or more hydrogens attached to a carbon or nitrogen. Exemplary substituents include alkyl, alkylidenyl, alkylcarboxy, alkoxy, alkenyl, alkenylcarboxy, alkenyloxy, aryl, aryloxy, alkylaryl, alkylaryloxy, —OH, amide, carboxamide, carboxy, sulfonyl, ═O, ═S, —NO₂, halogen, haloalkyl, fused saturated or unsaturated optionally substituted rings, —S(O)R, —SO₃R, —SR, —NRR′, —OH, —CN, —C(O)R, —OC(O)R, —NHC(O)R, —(CH2)ₙCO₂R or —(CH2)ₙCONRR′ where n is 0-4, and wherein R and R′ are independently H, alkyl, aryl or alkylaryl. Substituents also include replacement of a carbon atom and one or more associated hydrogen atoms with an optionally substituted heteroatom.

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include polypeptides containing post-translational modifications of the polypeptide, for example, glycosylations, acetylations, phosphorylations, and sulphations. In addition, protein fragments, analogs (including amino acids not encoded by the genetic code, e.g. homocysteine, ornithine, D-amino acids, and creatine), natural or artificial mutants or variants or combinations thereof, fusion proteins, derivatized residues (e.g. alkylation of amine groups, acetylations or others esterifications of carboxyl groups) and the like are included within the meaning of polypeptide.

The terms “substrate” and “support” are used interchangeably and refer to a material having a rigid or semi-rigid surface.

“Multiplexing” herein refers to an assay or other analytical method in which multiple analytes can be assayed simultaneously.

“Optional” or “optionally” means that the subsequently described event or circumstance may or may not occur, and that the description includes instances where the event or circumstance occurs and instances in which it does not.

“Sulfonyl” refers to —S(O)₂R, where R is aryl, —C(CN)═C-aryl, —CH₂CN, alkylaryl, or amine.

“Thioamide” refers to —C(S)NHR, where R is alkyl, aryl, alkylaryl or hydrogen.

“Thioether” refers to —SR, where R is alkyl, aryl, or alkylaryl.

The Substrate

The substrate can comprise a wide range of material, either biological, nonbiological, organic, inorganic, or a combination of any of these, which can react with the substrate modifier. The substrate comprises or is treated to comprise reactive oxygens, reactive hydroxyl groups, and/or reactive hydrogens with which the substrate modifier can react. For example, the substrate may be a polymerized Langmuir Blodgett film, functionalized glass, Si, Ge, GaAs, GaP, SiO₂, SiN₄, modified silicon, paper, leather, cellulose, nitrocellulose, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonate, or combinations thereof.

Substrates can be planar crystalline substrates such as silica-based substrates (e.g. glass, quartz, or the like), or crystalline substrates used in, e.g., the semiconductor and microprocessor industries, such as silicon, gallium arsenide and the like. The substrate can be aluminum, alumina, a ferrous material, or any reactive metallooxide. The substrate can be a zeolite or a nanoporous, microporous, or mesoporous silica (Zhao et al., Science 279:548, 1998; Zhao et al., J. Am. Chem. Soc. 120:6024-6036, 1998; Margolese et al., Chem. Mater. 12:2448-2459, 2000). The substrate may have a high surface area, such as silica and other metallooxides.

Silica sol-gels and aerogels can also be used as substrates, and can be prepared by methods known in the art. Aerogel substrates may be used as free standing substrates or as a surface coating for another substrate material.

The substrate can take any form such as lamellar, spherical or planar, and typically is a plate, slide, bead, pellet, disk, wafer, cover slip, particle, strand, precipitate, optionally porous gel, sheets, tube, sphere, container, capillary, pad, slice, film, chip, CCD chip, multiwell plate or dish, optical fiber, etc. The substrate can be a monolith, and can be a functional structure such as a plug.

The substrate may contain raised or depressed regions on which a histidine-containing protein can be located. The surface of the substrate can be etched using well known techniques to provide for desired surface features, for example trenches, v-grooves, mesa structures, or the like.

Surfaces on the substrate can be composed of the same material as the substrate or can be made from a different material, and can be coupled to the substrate by chemical or physical means. Such coupled surfaces may be composed of any of a wide variety of materials, so long as the surface has reactive groups which can be derivatized as taught herein.

The substrate and/or its optional surface are chosen to provide appropriate optical characteristics for the synthetic and/or detection methods used. The substrate and/or surface can be transparent to allow the exposure of the substrate by light applied from multiple directions. The substrate and/or surface may be provided with reflective “mirror” structures to increase the recovery of light for maximization of emission collected therefrom.

The substrate and/or its surface is generally resistant to, or is treated to resist, the conditions to which it is to be exposed, and can be optionally treated to remove any resistant material after exposure to such conditions.

Still further techniques include bead based techniques such as those described in PCT US/93/04145 and pin based methods such as those described in U.S. Pat. No. 5,288,514.

Additional flow channel or spotting methods applicable to patterned deposition of reactants and/or proteins are described in U.S. patent application Ser. No. 07/980,523, filed Nov. 20, 1992, and U.S. Pat. No. 5,384,261. Reagents are delivered to the substrate by either (1) flowing within a channel defined on predefined regions or (2) “spotting” on predefined regions. A protective coating such as a hydrophilic or hydrophobic coating (depending upon the nature of the solvent) can be used over portions of the substrate to be protected, sometimes in combination with materials that facilitate wetting by the reactant solution in other regions. In this manner, the flowing solutions are further prevented from passing outside of their designated flow paths.

Typical dispensers include a micropipette optionally robotically controlled, an ink-jet printer, a series of tubes, a manifold, an array of pipettes, or the like so that various reagents can be delivered to the reaction regions sequentially or simultaneously. Stamping techniques of any type can be used for depositing any of the components used in the methods taught herein.

Additionally, all forms of lithography can be used for patterned deposition, including for example photolithography, contrast enhancement lithography, electron beam lithography and soft lithography (Deng et al., Anal. Chem. 72(14):3176-80, 2000; Yang et al., Science 287:465-8, 2000; McDonald et al., Electrophoresis 21:27-40, 2000; Kane et al., Biomaterials 20:2363-76, 1999). For example, PMMA spin coated silicon wafers can be cut via electron beam lithography. Nickel metal can be applied via vapor deposition. The PMMA can then be lifted off. RIE (reactive ion etching) can be used to create nickel capped silicon features. Electron beams can be used to create 30 nm features with unlimited pattern capability. Master molds can be created, and stamp patterns can be formed by direct liquid or pattern transfer.

The Substrate Modifier

The substrate modifier is a molecule comprising a silane, a linker, and at least one active site. The silane comprises at least one leaving group such as an alkoxy group, a halogen (preferably chlorine), or an aryloxy group, and may contain two or three leaving groups, which may be the same or different (Crompton). Preferably the leaving group is an alkoxy group. Typically the silane comprises three leaving groups, for example trimethoxysilane or triethoxysilane, allowing for three points of attachment to the substrate.

The substrate modifier can be of low molecular weight and/or generally linear structure to allow a high degree of surface derivatization. This can result not only in a high degree of incorporation of chelating groups onto the substrate, but also can limit the number of unreacted reactive groups on the substrate which might subsequently adversely affect protein structure or other aspects of the desired technique.

Examples of spacers or linkers are optionally substituted polyethyleneglycols, dicarboxylic acids, polyamines, straight, branched, and/or cyclic alkyls, aryls, alkylaryls, and combinations thereof. The linker preferably is inert and does not cause side reactions with the subsequent chemistry used. The linker may include one or more polymerizable groups, which can be used to form crosslinks between individual linkers after reaction of the substrate modifier with the substrate.

The active site is chosen so that it does not react with the substrate in the synthetic scheme employed. The active sites are optionally protected initially by protecting groups compatible with the synthesis scheme being used. Exemplary active sites include amine groups and glycidyl ethers. Among a wide variety of protecting groups which are useful are FMOC, BOC, t-butyl esters, t-butyl ethers, and the like. Various exemplary protecting groups are described in, for example, Atherton et al., Solid Phase Peptide Synthesis, IRL Press (1989). Preferred linkers are lower alkyls.

Nonlimiting examples of the substrate modifier include glycidoxypropyltrimethoxysilane, N,N′-bis-(trimethoxysilylpropyl)ethylene diamine, and halomethylphenyl (e.g., cloromethylphenyl, iodomethylphenyl) derivatives of silane.

In one aspect, the invention provides a substrate modifier having the formula:

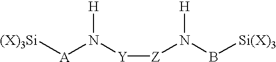

wherein each X on a given Si is independently selected from alkyl, aryl, hydroxy, alkoxy, aryloxy, halo, wherein at least one X on a given Si is a leaving group selected from alkoxy, halo, hydroxy and aryloxy; A and B are linkers as described above, including mixtures thereof, may be the same or different, may be branched, linear, cyclic, or combinations thereof, and typically comprise from one to 20 atoms in the most direct connection between the proximal nitrogen and silicon atoms; and Y and Z form a two or three carbon alkyl, aryl or alkylaryl group. Preferably two silane groups are included, but one silane group and its associated linker can optionally be deleted. Preferably the silane groups are bound to three leaving groups. Preferably Y and Z form an ethylene group.

Incorporation of the Substrate Modifier

The substrate modifier can be incorporated onto or into the substrate in a variety of ways. The substrate modifier may be reacted with reactive species on the surface of a preexisting substrate, and thereby form a surface-derivatized substrate. This can be done at ambient temperatures or can be accelerated by raising the temperature. The substrate is preferably moist to permit hydrolysis to occur. Trialkoxy silanes can react with active oxygens and/or active hydroxyls on the substrate via a condensation reaction. Reaction schemes can be used with a leaving group on the substrate as well, for example a halide, preferably a chloride; the substrate modifier can be reacted via hydrosilation to such a substrate, or via a Grignard or other organometallic reagent. The substrate modifier can also be introduced via reaction of a disilane bound to a linker bound to an active site; the disilane reacts with hydroxyl group(s) on the substrate to form an —O—Si— bond between the linker and the substrate. The substrate modifier can be incorporated into the substrate itself as it is formed. For example, a diamine such as N,N′-bis-(trimethoxysilylpropyl)ethylene diamine can be incorporated during polymerization of tetraethoxysilane to silica. Incorporation of the substrate modifier produces an activated substrate suitable for incorporation of a reagent via the active site.

Conversion of the Activated Substrate into a Chelating Substrate

Conversion of the activated substrate into a chelating substrate is performed via derivatization of the active site with at least one reagent. The reagent must comprise a functional group that can react with the active site and also comprises or can be modified to comprise a ligand suitable for coordinating a metal ion that can coordinate a histidine residue. Exemplary reagents include bis-carboxymethyl-L-lysine trisodium salt, and a haloacetic acid such as bromoacetic acid, which can also be used to modify bis-carboxymethyl-L-lysine. The active site itself can form part of the chelating functionality, or the chelating functionality can be introduced entirely via the reagent(s). Preferably the functional group and the active site will not react with other components of the system, including the surface of the substrate, the metal ion to be chelated, or the protein to be bound to the metal chelate.

The ligand is one that can coordinate with a hexavalent metal ion, and may be present in the reagent in an inactive form. The inactive form may be converted to an available ligand by either removal of a blocking group preventing access to the ligand, by addition of an additional reagent which adds an additional functionality necessary for chelation of the metal ion, or a combination thereof.

Preferred chelators are of a form which can chelate a hexavalent metal ion, for example, cobalt, nickel, copper, and zinc, in a tetracoordinate fashion, leaving two available coordination sites on the metal ion. These available sites must be available in a cis orientation, at least transiently, in order for the histidine-containing protein to bind. Typical groups used to chelate the metal ion include those with available electrons, including carboxylate and amine groups. Cobalt and copper are preferred to nickel for uses in which less reversible binding of the histidine-containing protein is desired for maintaining protein localization, while nickel is preferred for uses in which reversible binding of histidine-containing proteins is desirable.

The chelating functionality can be in the form of a nitrilotriacetic acid. The chelating functionality can be in the form of a biscarboxymethyl ethylene or propylene diamine. Nonlimiting examples of the chelator include derivatized bis-carboxymethyl-L-lysine and N, N′-bis-(silylpropyl)-N,N′-bis-(carboxymethyl)ethylene diamine.

After attachment of the chelator, a metal ion suitable for binding a histidine-containing protein is bound to the chelator, for example nickel, zinc, cobalt or copper.

The Histidine-Containing Protein

The protein can be any protein which contains, either naturally or via manipulation, histidines of a number and arrangement that can bind to the two available cis valencies on the tetracoordinated metal ion linked to the chelating substrate. Naturally occurring proteins of this type include metal-binding proteins such as Helicobacter pylori nickel binding protein (U.S. Pat. No. 5,780,040 issued Jul. 14, 1998 to Plaut et al.). Systems for introducing histidine tags into recombinantly produced proteins are commercially available (Qiagen). Typical histidine tags incorporated into recombinantly produced proteins include 2×His, 4×His and 6×His tags, as described in U.S. Pat. Nos. 5,284,933 (issued Feb. 8, 1994 to Dobeli et al.) and 5,310,663 (issued May 10, 1994 to Dobeli et al.).

The protein can be synthetically made, can be recombinantly expressed in a prokaryote, eukaryote, or archeon, or can be purified from a nonrecombinant source. The source from which the protein is obtained may be subjected to various preparative steps to isolate and/or purify the protein prior to binding the protein to the chelating surface; a number of preparative steps are known in the art.

The protein may have a known or unknown function. The protein can be, for example, a signaling molecule, an enzyme, a light-emitting molecule, a receptor, a signal transduction molecule, an ion channel, an antibody, or a protein identified by genomic sequencing and analysis. Exemplary enzymes include silicateins, alkaline phosphatase, horseradish peroxidase, β-galactosidase, glucose oxidase, a bacterial luciferase, an insect luciferase and sea pansy luciferase (Renilla koellikeri).

Exemplary light-emitting proteins include “green fluorescent protein” (GFP), which refers to both native and mutated versions of Aequorea green fluorescent protein that have been identified as exhibiting altered fluorescence characteristics, including altered excitation and emission maxima, as well as excitation and emission spectra of different shapes (Delagrave, S. et al. (1995) Bio/Technology 13:151-154; Heim, R. et al. (1994) Proc. Natl. Acad. Sci. USA 91:12501-12504; Heim, R. et al. (1995) Nature 373:663-664). Delgrave et al. isolated mutants of cloned Aequorea victoria GFP that had red-shifted excitation spectra (“RFP”). Bio/Technology 13:151-154 (1995). Heim, R. et al. reported a mutant (Tyr66 to His) having a blue fluorescence (Proc. Natl. Acad. Sci. (1994) USA 91:12501-12504).

Silicateins (silica proteins) are enzymes related to the cathepsin L and papain family of proteases which are involved in the polymerization of silica in marine organisms (Shimizu et al., Proc. Natl. Acad. Sci. USA 95:6234-6238, 1998). Silicateins isolated from the sponge Tethya aurantia include silicatein alpha, beta and gamma, which form a central protein filament within the sponge spicule. These protein filaments and the individual subunits have been shown to cause the deposition of silica and silicone in vitro when supplied with appropriate substrates (Cha et al., Proc. Natl. Acad. Sci. USA 96:361-365, 1999). The methods taught herein include methods of attaching silicateins to substrates on which they do not naturally occur, and employing the enzymatic activity of the silicateins to deposit silica and/or silicone thereon.

Silicatein alpha, silicatein beta and RFP have separately been recombinantly expressed and used herein in modified form incorporating 6xHis histidine tags.

Binding the Protein to the Substrate

The histidine-containing protein can be bound to the substrate by incubation in solution with the chelating substrate. The methods taught herein for forming and localizing the chelators on the substrate allow a degree of control previously unobtainable for binding and localizing proteins. Patterned deposition of the protein can be controlled by the patterned deposition of any of the components used, for example a material used to activate the surface of the substrate, the substrate modifier, the reagent used to form the chelator, or the protein itself. The activity of the protein can be exhibited, where desired, by subjecting the protein-bound substrate to appropriate conditions for the enzymatic or other activity of the protein.

Kits

Kits comprising reagents useful for performing the methods of the invention are also provided. Typically, a kit comprises: (1) a substrate modifier and (2) a reagent, each of which is retained by a housing. The kit can optionally contain a substrate. Instructions for using the kit to perform a method of the invention are provided with the housing, and may be located inside the housing or outside the housing, and may be printed on the interior or exterior of any surface forming the housing which renders the instructions legible. The kit may be in multiplex form. The substrate may be a microarray.

### EXAMPLES

The following examples are set forth so as to provide those of ordinary skill in the art with a complete description of how to make and use the present invention, and are not intended to limit the scope of what is regarded as the invention. Efforts have been made to ensure accuracy with respect to numbers used (e.g., amounts, temperature, etc.) but some experimental error and deviation should be accounted for. Unless otherwise indicated, parts are parts by weight, temperature is degree centigrade and pressure is at or near atmospheric, and all materials are commercially available.

### Example 1

6×-His tagged proteins were attached to flat surfaces via sequential growth of an NTA-type linker employing (1) glycidoxyproplytrimethoxysilane, (2) bis-carboxymethyl-L-lysine trisodium salt, (3) coordination of Ni2+salts (or Co2+ or Cu2+) and finally (4) attachment of the 6×-His tagged protein. Pattering the initial silane on the surface of interest permitted subsequent attachment of the proteins in the identical pattern. The bis-carboxymethyl-L-lysine trisodium salt was prepared by first protecting the epsilon amine, the reacting the primary alpha amine with two equivalents of bromoacetic acid, and then forming the trisodium salt from the three carboxyl groups; the epsilon amine is then deprotected and reacted with the gycidyl ether. Two proteins (silicatein alpha and ‘red fluorescent protein—RFP’) were attached in this way to both glass and silicon substrates. The primary question when binding proteins to substrates is the retention of their critical protein properties. This was ensured by observing that, for RFP, red fluorescence (which is folding specific) was still observed after binding to the substrate. In the case of silicatein-alpha, proper folding and function of the protein was demonstrated by observing its catalytic activity of hydrolysis and subsequent polymerization of TEOS to amorphous silica at pH=7.

### Example 2

Previously it was not possible to attach proteins to mesoporous silicas such as the SBA series without functionalizing and derivatizing the silica surfaces after formation. This is due, in part, to the pH of the hydrolysis and condensation reactions (pH<2.0 for SBA mp-SiO2) which would interfere with known protein binding techniques such as NTA chemistry. To this end, we developed a method of incorporating a flexible tetracoordinate complex into mesoporous silica that chelates Ni2+ and Co2+metal ions in a similar fashion. Incorporation of the initial active sites during the mesoporous synthesis allows high loading of active sites. The polar nature of these sites directs their presence to the polar surfactant interface and ensures high availability in the final cured mesoporous silica after surfactant removal. The functional mesoporous material was prepared by mixing TEOS with N,N′-bis(trimethoxysilylpropyl)ethylene diamine under normal mesoporous silica conditions at <pH=2.0. After isolation and extraction of the surfactant by washing, the bridging ethylene diamine groups were reacted with bromoacetic acid to prepare the four-coordinate complex, which subsequently was bound to Ni2+salts. The reaction is depicted on the next page. This complex, integrally attached to the mesoporous silica, was then used to bind 6×-His tagged proteins in a fashion analogous to the NTA-type surfaces described in Example 1.

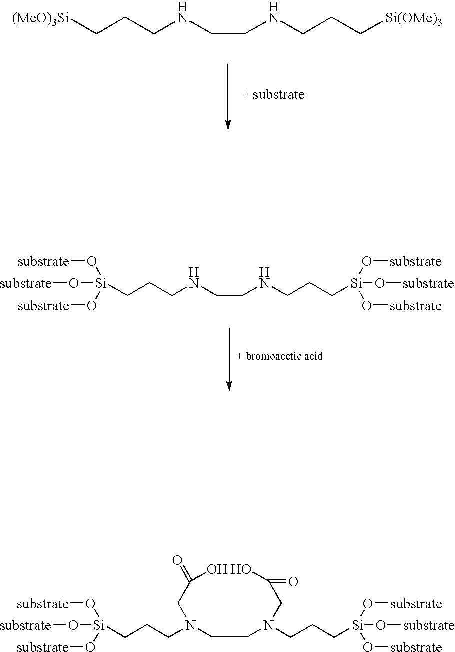

### Example 3

Results from the localization of RFP, silicatein alpha and silicatein beta to various substrates and their use to respectively emit light or deposit silica or silicone are shown in the attached figures.

Although the invention has been described in some detail with reference to the preferred embodiments, those of skill in the art will realize, in light of the teachings herein, that certain changes and modifications can be made without departing from the spirit and scope of the invention. Accordingly, the invention is limited only by the claims.

What is claimed is:

1. A method of forming an activated substrate, comprising:
providing a substrate comprising active oxygen atoms, active hydroxyl groups, alkoxy groups, halogens or a combination thereof,
providing a substrate modifier having the formula 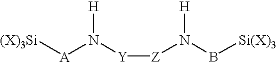
wherein each X on a given Si is independently selected from alkyl, aryl, hydroxy, alkoxy, aryloxy, halo, wherein at least one X on a given Si is a leaving group selected from alkoxy, halo, hydroxy and aryloxy; A and B are linkers selected from optionally substituted polyethyleneglycols, dicarboxylic acids, polyamines, alkyls, aryls, alkylaryls, and combinations thereof, may be the same or different, and may be branched, linear, cyclic, or combinations thereof, and Y and Z form a two or three carbon alkyl, aryl or alkylaryl group; and
reacting the substrate modifier with the substrate by a condensation reaction to form an activated substrate.

2. A method of forming a chelating substrate, comprising:
performing the method of claim 1;
providing a reagent that is a haloacetic acid;
reacting the reagent with the activated substrate to form a chelator; and
binding a metal ion selected from cobalt, nickel, copper and zinc to the chelator to form a tetracoordinate metal chelate with two available cis valencies, thereby converting the substrate into a metal-chelated substrate.

3. A method of depositing a protein on a substrate, comprising:
performing the method of claim 2;
providing a histidine-containing protein wherein the number and location of the histidine residues within the protein allow binding of the protein to the two cis valencies on the metal chelate; and
incubating the histidine-containing protein with the metal-chelated substrate so that the histidine-containing protein binds via the histidine residues to the metal chelate to form a protein-substrate complex.

4. The method of claim 3, wherein the protein-substrate complex forms a pattern on the substrate.

5. The method of claim 4, wherein the pattern is formed by stamping.

6. The method of claim 4, wherein the pattern is formed by photolithography.

7. The method of claim 4, wherein the pattern is formed by soft lithography.

8. The method of claim 4, wherein the pattern is formed by electron beam lithography.

9. The method of claim 4, wherein the pattern is formed by patterned deposition of the substrate modifier.

10. The method of claim 4, wherein the pattern is formed by patterned deposition of the reagent.

11. The method of claim 4, wherein the pattern is formed by patterned deposition of the histidine-containing protein.

12. The method of claim 4, wherein the pattern is formed by patterned activation or exposure of the substrate surface prior to reaction with the substrate modifier.

13. An activated substrate produced by the method of claim 1.

14. A metal-chelated substrate produced by the method of claim 2.

15. A protein-substrate complex produced by the method of claim 3.

16. A sensor comprising the protein-substrate complex of claim 15.

17. A biocatalyst comprising the protein-substrate complex of claim 15.

18. A microfluidic system comprising the protein-substrate complex of claim 15.

19. The protein-substrate complex of claim 15, wherein the histidine-containing protein is a green fluorescent protein.

20. The protein-substrate complex of claim 15, wherein the histidine-containing protein is a silicatein.

21. The protein-substrate complex of claim 20, wherein the silicatein is silicatein alpha.

22. The protein-substrate complex of claim 20, wherein the silicatein is silicatein beta.

23. A method of depositing silica or silicone on a substrate, comprising:
forming the protein-substrate complex of claim 20;
contacting the protein-substrate complex with an optionally derivatized alkoxysilane, wherein the alkoxysilane is optionally derivatized with one or more optionally substituted alkyl groups,
wherein said contacting is performed under conditions suitable for said silicatein to polymerize said optionally derivatized alkoxysilane to form a silica- or silicone-derivatized substrate.

24. The method of claim 22, wherein the optionally derivatized alkoxysilane is tetraethoxysilane.

25. The method of claim 22, wherein the optionally derivatized alkoxysilane is phenyltriethoxysilane.

26. The method of claim 22, wherein the optionally derivatized alkoxysilane is methyltriethoxysilane.

27. The method of claim 1, wherein the substrate modifier is reacted with the substrate after the substrate is formed.

28. The method of claim 1, wherein the substrate modifier is reacted with the substrate as the substrate is being formed.

29. The method of claim 1, wherein the substrate modifier is N, N′-(trimethoxysilylpropyl)ethylene diamine.

30. The method of claim 2, wherein the reagent is bromoacetic acid.

31. The method of claim 2, wherein the metal ion is a cobalt ion.

32. The method of claim 2, wherein the metal ion is a copper ion.

33. The method of claim 2, wherein the metal ion is a nickel ion.

34. A kit comprising:
a substrate modifier comprising a silane, a linker and an active group;
a substrate comprising a reactive species that can react with the silane;
a housing for retaining the substrate modifier and the substrate;
instructions provided with said housing that describe how to use the components of the kit to link the substrate modifier to the substrate.

35. The kit of claim 34, further comprising a reagent comprising a functional group and a ligand for a metal ion.
